(19) United States
(12) Patent Application Publication
(10) Pub. No.: US 2010/0330137 A1
(43) Pub. Date: Dec. 30, 2010

(54) HIGH BIOAVAILABILITY FORMULATION OF MANGOSTEEN AND MANUFACTURING PROCESS THEREOF

(76) Inventor: Mauro Mantovani, Milano (IT)

Correspondence Address: DICKSTEIN SHAPIRO LLP, 1825 EYE STREET NW, Washington, DC, 20006-5403, US

(21) Appl. No.: 12/865,968

(22) PCT Filed: Mar. 10, 2008
(86) PCT No.: PCT/EP2008/002004

§ 371 Date: Aug. 3, 2010

(51) Int. Cl. A61K 36/38; A61K 9/16; A61K 8/02; A61P 17/00; A61P 29/00; A61P 31/00; A61P 17/18; A61Q 19/00; A61Q 90/00; A23L 1/28; A23P 1/02; A23P 1/04; A23P 1/00; B02C 19/00

(52) U.S. Cl. 424/401; 424/769; 424/499; 424/493; 424/115; 426/648; 426/285; 366/348; 426/52; 241/28

(57) ABSTRACT

The present invention relates to a pharmaceutical or dietary composition containing Mangosteen (Garcinia Mangostana pericarp) and being characterized by high bioavailability. A process for the manufacture of this composition is a further object of the present invention. In particular, the present invention relates to a composition containing Mangosteen extract and oligosaccharides in an amount ranging from 5 to 50 w/w % with respect to the Mangosteen extract or a fermented Mangosteen product and to a process for obtaining thereof.

### FIELD OF THE INVENTION

The present invention relates to a pharmaceutical or dietary composition containing Mangosteen (Garcinia Mangostana pericarp) and being characterized by high bioavailability. A process for the manufacture of this composition is a further object of the present invention.

### BACKGROUND ART

The Mangosteen is a fruit originating in Southeast Asia. The latin name for the Mangosteen is Garcinia mangostana Linnaeus. The tree grows from 7 to 25 meters tall. The rind (pericarp) of the edible fruit is deep reddish purple when ripe.

The edible internal part is normally used to produce an appreciated juice.

In Southeast Asia the pericarp is used as a traditional medicine. The sliced and dried rind is powdered and administered to overcome dysentery. While formulated into an ointment, it is applied on eczema and other skin disorders. The rind decoction is taken to relieve diarrhea and cystitis, gonorrhea and gleet and is applied externally as an astringent lotion. Filipinos employ a decoction of the leaves and bark as a febrifuge and to treat thrush, diarrhea, dysentery and urinary disorders.

Many scientific studies show that the pericarp of Mangosteen is reach in active compound of different groups, such as:

Xanthones (α- and γ-mangostin, Garcinone E, etc.)

Flavonoids

Cathequines

Polyphenols

Tannins

The biological effect of Mangosteen was tested in vitro mainly as antioxidant, antihistaminic, anti-inflammatory and anti-microbial. This is in accordance with the traditional use.

Nowadays, the Mangosteen is commercialized all over the world as a food supplement.

In particular, the whole fruit juice as well as the pericarp dry extract formulated in solid oral forms, like tablets and capsules, can be found on the market.

In general, the dry extract available on the market are valued on the base of α- and γ-mangostin content, that is considered the most important and specific active component.

However, the food supplements available on the market are characterized by low bioavailability.

The inventor of the present invention has experimentally verified that the bioavailability of α- and γ-mangostin is close to zero.

There are three factors that can decrease the availability of Mangosteen active compounds:
1) The α- and γ-mangostin is sensible to low pH and it is partially inactivated in the stomach;
2) As for many other natural active ingredients, it is known that the immediate release causes a high peak concentration in intestinal lumen. This can saturate the active and passive transport system and that causes a decrease of blood level and bioavailbility of α- and γ-mangostin;
3) During the extraction of mangostin, many of other active compounds are discharged. The secondary classes of bioactives, like flavonoids, cathequines, polyphenols and tannins, are on the contrary essential for the synergism with the xanthones, both for the absorbtion in the body and also for the biological effects.

### DESCRIPTION OF THE INVENTION

The present invention relates to an innovative formulation and process in order to increase the bioavailability of Mangosteen components, thus solving the problems that characterise the actual Mangosteen extracts on the market.

The invention may comprise four different innovative features that are, independently from one another, apt to achieve an inventive result and that, when used all together, are synergistic, as will be explained below.

A first aspect of the invention is based on the idea to ferment the Mangosteen and add the said fermented Mangosteen product, preferably in a percentage from 5% to 50% w/w, to the normal Mangosteen extract. The Mangosteen fruit is in fact a substrate suitable for fermentation. In the meaning of the present invention, the term “fermented Mangosteen product” is the product obtainable by the fermentation of a Mangosteen fruit as described below.

The Mangosteen is fermented using as a starter Saccaromyces Boulardii or an analogue microrganism. Unexpectedly, the inventor found that the fermented Mangosteen product, as it is or supported in maltodextrin, increases dramatically the bioavailability of xanthones present in the preparation. This is due to the formation, during fermentation, of natural oligosaccharides, the most represented in the class being 1,3-1,6-β-D-glucane. These components may enhance the absorption of xanthones, as carrier in the gastroenteric tract and, from a biological point of view, increase the effect of xanthones with mechanism of immunostimulation.

The oligosaccharides can also be added to the formulation as further ingredient or in replacement of the fermented Mangosteen product in w/w % of between 5% and 50%.

Even after the enhancement of bioavailability described above, the problem of disruption of active ingredients in the stomach acid ambient still remains. For this reason, a further object of the present invention is to provide the above composition with a gastroresistant coating, obtained by covering the Mangosteen extract beadlets with gastroresistant layers, preferably of natural origin as vegetable waxes (carnauba wax, sandarac tree gum, arabic gum, shellac) or other natural layers like keratin in a percentage w/w from 5% to 30%. A good protective action was obtained also with synthetic coating agents, like cellulose acetophtalate, acrylic and meta-acrylic polymers, polyvinilacetate-phtalate and others.

The present invention also addresses the problem of high peak concentration, that can saturate the active and passive transport systems of intestinal surface, causing a low absorption of active ingredients present in the Mangosteen extract.

It is a further object of the invention either i) the inclusion of a Mangosteen extract in a matrix composed by preferably natural or synthetic polymers in percentage from 20% to 80%, these polymers being preferably selected from: hydroxy-propyl-methylcellulose, starch, methylcellulose, alginates, gelatine; or ii) the coating of the Mangosteen extract with such natural or synthetic polymers, for example by spraying in a fluid bed. The matrix obtained there from delivers slowly the bioactive molecules of Mangosteen in the gastro-enteric tract, thus avoiding the saturation of transport systems through the intestinal membrane. The final result is an important enhancement of the bioavailability of the active molecules of Mangosteen.

In order to obtain the best results, the inventor found that also the manufacturing process is crucial. Therefore, a further object of the invention is a method of preparing the inventive composition that generated unexpectedly positive results.

According to a first aspect, the process comprises a step of mixing a Mangosteen extract with a fermented Mangosteen product in order to obtain the composition of the invention.

Preferably, both the Mangosteen extract and the fermented Mangosteen product are granulated. More preferably, they have a granulometry from 20 to 45 mesh or better about 40 mesh.

The granulated composition may be used as such or the process may further comprise a step of formulating the inventive composition into tablets, capsules or sachet fillings, according to techniques well known to the skilled artisan, such as those described in Remington's Pharmaceutical Sciences Handbook, Mack Pub. Co., N.Y., USA, 17th edition, 1985. Other conventional pharmaceutical or dietary formulations can be used as well as according to specific needs.

Mangosteen extract can be obtained according to conventional techniques well known to the skilled artisan. Mangosteen extract is also available on the market.

The fermented Mangosteen product can be obtained by a fermentation process that comprises fermenting whole fruits of Mangosteen, preferably homogenized in advance, with a starter, preferably Saccaromyces Boulardii, at a temperature of 35-55° C. for 2-4 months.

The flow sheet of a preferred process is shown in FIG. 1 and may comprise the following steps:
1) granulation of Mangosteen extract with preferably natural or synthetic cellulose derivatives in percentage from 10% to 80%, preferably 15% to 50%, wherein such cellulose derivatives are preferably selected from: hydroxyl-propyl-methylcellulose, starch, methylcellulose, alginates, gelatine; or alternatively granulating Mangosteen extract and coating the beadlets so obtained with natural or synthetic cellulose derivatives in percentage from 10% to 80%, preferably from 15% to 50%, wherein such cellulose derivatives are preferably selected from: hydroxyl-propyl-methylcellulose, starch, methylcellulose, alginates, gelatine;
2) Coating of the granules obtained according to step 1) with gastroresistant agents preferably of natural origin, as vegetable waxes (carnauba wax, sandarac tree gum, arabic gum, shellac) or other natural layers like keratin, in a percentage w/w from 5% to 30%; or with synthetic coating agents, like cellulose acetophtalate, acrylic and meta-acrylic polymers, polyvinilacetate-phtalate and others;
3) Adding of fermented Mangosteen, as it is or supported in maltodextrines, to the coated granules and mixing properly;
4) Adding the composition with direct compression excipients like microcrystalline cellulose, magnesium stearate, talc, lactose, in percentage w/w between 3% and 20%, preferably around 15%, and mixing properly;
5) reducing the powder, for example by direct compression, into tablets or capsule filling or sachet filling.

Alternatively, steps 1) and 2) can be combined in one step of granulating Mangosteen extract with preferably natural or synthetic cellulose derivatives and with gastroresistant agents preferably of natural origin, wherein the preferred cellulose derivatives and the preferred gastroresistant agents as well as their amounts employed in the composition are those depicted in steps 1) and 2) above.

Preferably, the product of steps 1) to 3) will have finally a granulometry of between 20 and 45 mesh. If such product, after the coating steps, has an higher granulometry, a milling step according to conventional techniques can be provided to adjust the beadlets granulometry to the preferred range above.

A variant to the above process (FIG. 2) is to include the fermented Mangosteen in the matrix of step 1) together with Mangosteen extract (thus combining steps 1) and 3)), and after that, applying the coating process according to step 2).

Another variant of the process (FIG. 3) comprises the direct compression of granules of step 1) or of the combination of steps 1) and 3), as described above, followed by the milling of the tablets obtained there from and then coating the dry particles according to step 2).

According to another variant of the process, step 3) of adding fermented Mangosteen in any of the above variants of the process is replaced by a step of adding oligosaccharides as such, preferably 1,3-1,6-p-D-glucane, to the other components.

### BRIEF DESCRIPTION OF THE DRAWINGS

FIG. 1 shows the flow sheet of the process for manufacturing the composition of the invention;

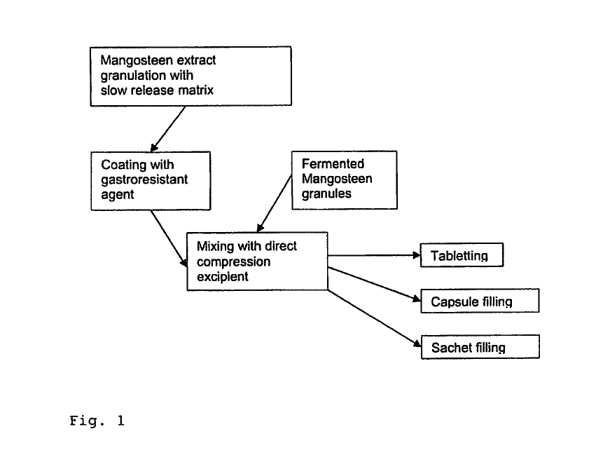

FIG. 2 shows the flow sheet of a first variant of the process of FIG. 1;

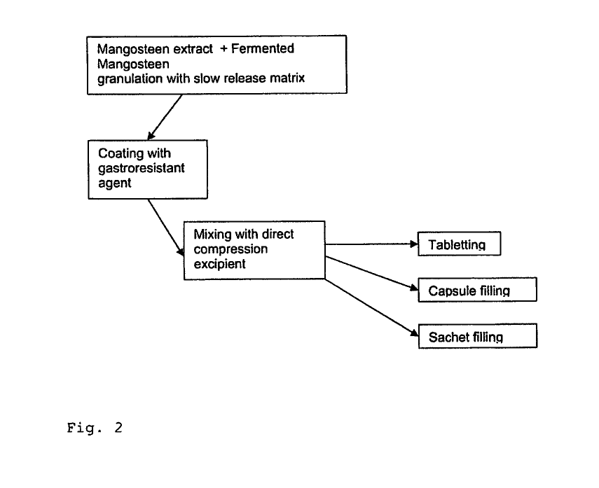

FIG. 3 shows the flow sheet of a second variant of the process of FIG. 1.

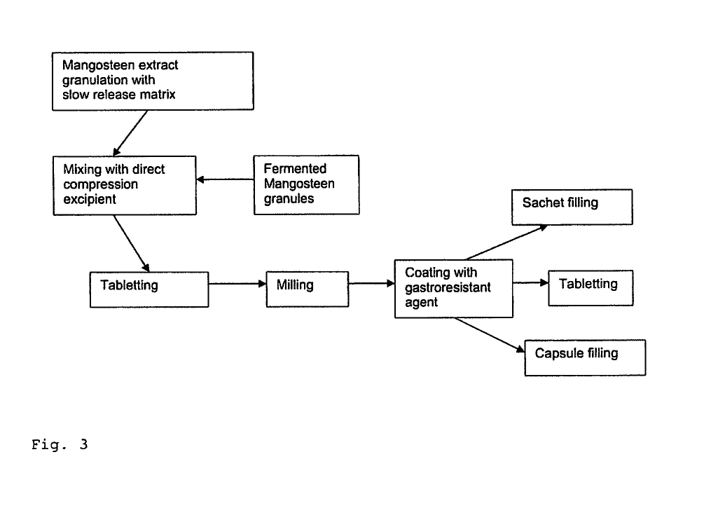

### EXPERIMENTAL PART

Herein below it is described an example of formulation (tablets), obtained according to the process of the present invention. The results obtained in term of bioavailability, in comparison to other two formulations available on the market, are also showed.

### Example of Preparation 1

200 gr of Mangosteen extract (standardised at 20 w/w % in xanthones) is coated by spraying in a fluid bed with a solution of hydroxypropyl methyl cellulose at 50-60 w/w % in water up to a content of hydroxypropyl methyl cellulose of 15 w/w %. Beadlets of 50/60 mesh are thus obtained.

Such beadlets are then coated, by spraying in a fluid bed, with a solution of carnauba wax at 50-60% w/w % in a non-polar solution, until a 15 w/w % of carnauba wax is reached. The beadlets are milled in order to adjust the granulometry to about 40 mesh.

A 20 w/w % of fermented Mangosteen product (obtained as reported below) having the same granulometry of about 40 mesh is thus added, together with a 5 w/w % of direct compression excipient (magnesium stearate) and the powder is finally formed by direct compression into tablets.

Preparation of Mangosteen extract: A hydroalcoholic solution of Mangosteen pericarp is extracted in a soxhlet extractor at 60-80° C. for 5, 6 times. Each extraction step lasts 4 to 6 hours. The solution is then concentrated under vacuum up to dryness. The residue is treated under vacuum at about 60-70° C., to obtain solid particles that are milled to a granulometry of about 50 mesh.

### Example of preparation 2

Micropellets are obtained by granulating the following components (w/w %):

Mangosteen extract (20% in xanthones)=50%

Hydroxypropylmethylcellulose (matrix) 30%

Carnauba wax (coating agent)=20%

A mixture of 80 w/w % of these micropellets and 20 w/w % of micropellets obtained by 50 w/w % of fermented Mangosteen and 50 w/w % of Maltodextrin (support) is then obtained and the resulting micropellets are coated with microcrystalline cellulose and magnesium stearate (85 w/w % of micropellets/14 w/w % microcrystalline cellulose/1 w/w % magnesium stearate)

Tablets of 500 mg each are thus obtained by means of conventional direct compression technology.

Fermentation Process

Harvested Mangosteen whole fruits are homogenized and the mixture is put into a fermentation reactor and is added with a microbic starter, namely Saccharomyces boulardii. The temperature is brought to 35-55° C. in order to start the fermentation, that is then kept for about 3 months or more.

The resulting pale-white biomass is thick like a syrup.

The biomass can be added with maltodextrin (about 50 w/w %) and then granulated up to a granulometry of about 40 mesh.

Bioavailability Tests

Materials and Methods

The bioavailability was tested in No. 3 human subjects, two males and a female. Tablets of 500 mg, produced using the technology described in the present invention with a formulation as described in the example above, were used.

The two reference products used to compare the bioavailability were respectively:

Reference A: Mangosteen dry extract 40% in xanthones: dose 250 mg (in 500 mg tablet)

Reference B: Mangosteen dry extract 95% in xanthones: dose 100 mg (in 500 mg tablet)

The products were administered as single dose, at different times, with an appropriate period of wash-out between one administration and the next one. The blood concentration of α/γ-mangostin was tested by High Pressure Liquid Chromatography after the following times: 1, 2, 3, 4 hours.

Results

The results are summarised in the following table, as average of three subjects.

TABLE 1

| Product | 1 h | 2 h | 3 h | 4 h |
|---|---|---|---|---|
| Test | 0.40 μg/ml | 0.60 μg/ml | ND | ND |
| (invention) | | | | |
| Reference A | 0.033 μg/ml | 0.060 μg/ml | ND | ND |
| (40% assay) | | | | |
| Reference B | 0.029 μg/ml | 0.033 μg/ml | ND | ND |
| (95% assay) | | | | |

ND = Not detectable

From the above data, it can be seen that the inventive formulation has a bioavailability that is about 11 times greater than the reference A composition and 16 times greater than the reference B composition.

The data indicate also a peak of concentration in the second hour.

This data demonstrate the utility and industrial application of the present invention.

The composition of the invention can be used for veterinary or pharmaceutical use and in particular for use as antioxidant, antihistaminic, anti-inflammatory and anti-microbial. The composition of the invention can also be used in a cosmetic formulation.

The composition can also be used as a food or dietary supplement for humans or animals.

1. Composition containing Mangosteen extract and oligosaccharides in an amount ranging from 5 to 50 w/w % with respect to the Mangosteen extract.

2. Composition according to claim 1, wherein the said oligosaccharides are 1,3-1,6-β-D-glucane.

3. Composition containing Mangosteen extract and a fermented Mangosteen product in an amount of between 5 and 50 w/w % with respect to the Mangosteen extract.

4. Composition according to claim 1, the composition being in powder form and having a granulometry of between 20 and 45 mesh or about 40 mesh.

5. Composition according to claim 1, wherein the components are in particle form and the said particles comprise a gastroresistant additive.

6. Composition according to claim 5, wherein the said gastroresistant additive is of natural origin as vegetable waxes; or a wax selected from carnauba wax, sandarac tree gum, arabic gum, shellac or other natural layers; or keratin; or synthetic coating agents; or an agent selected from cellulose acetophtalate, acrylic and meta-acrylic polymers, polyvinilacetate-phtalate.

7. Composition according to claim 5, wherein the said gastroresistant additive is provided in a percentage w/w from 5% to 30% with respect to weight of the composition.

8. Composition according to claim 1, comprising natural or synthetic polymers apt to provide a slow release of the composition, or a polymer selected from: hydroxy-propyl-methylcellulose, starch, methylcellulose, alginates, gelatine.

9. Composition according to claim 8, the said polymers being provided in w/w percentage of between 20 and 80%.

10. Composition according to claim 5, wherein the particles of the Mangosteen extract are coated with at least a first layer of said natural or synthetic polymers apt to provide a slow release of the composition and optionally with at least a second layer of said gastroresistant additive.

11. Process for manufacturing the composition of claim 1, comprising a step of mixing a Mangosteen extract with a fermented Mangosteen product.

12. Process according to claim 11, wherein both the Mangosteen extract and the fermented Mangosteen product are granulated to a granulometry of 20-45 mesh or about 40 mesh.

13. Process according to claim 11, wherein the composition is formed into tablets, capsules or sachet fillings.

14. Process according to claim 11, comprising the following steps:
1) granulation of Mangosteen extract with preferably natural or synthetic cellulose derivatives in w/w percentage from 10% to 80%, or from 15% to 50%, wherein such cellulose derivatives are selected from: hydroxyl-propyl-methylcellulose, starch, methylcellulose, alginates, gelatine; or alternatively granulating Mangosteen extract and coating the particles so obtained with natural or synthetic cellulose derivatives in percentage from 10% to 80%, or from 15% to 50%, wherein such cellulose derivatives are selected from: hydroxyl-propyl-methylcellulose, starch, methylcellulose, alginates, gelatine;
2) Coating of the granules obtained according to step 1) with gastroresistant agents preferably of natural origin, as vegetable waxes (carnauba wax, sandarac tree gum, arabic gum, shellac) or other natural layers like keratin, in a percentage w/w from 5% to 30%; or with synthetic coating agents, like cellulose acetophtalate, acrylic and meta-acrylic polymers, polyvinilacetate-phtalate;
3) Adding of fermented Mangosteen, as it is or supported in maltodextrins, to the coated granules and mixing properly;
4) Adding the composition with direct compression excipients like microcrystalline cellulose, magnesium stearate, talc, lactose, in percentage w/w between 3% and 20%, or around 15%, and mixing properly;
5) reducing the powder into tablets or capsule filling or sachet filling.

15. Process according to claim 14, wherein steps 1) and 2) are combined in one step of granulating Mangosteen extract with preferably natural or synthetic cellulose derivatives and with gastroresistant agents preferably of natural origin, wherein the cellulose derivatives and the gastroresistant agents as well as their amounts employed in the composition are those depicted in steps 1) and 2).

16. Process according to claim 11, comprising the following steps:
1) granulation of Mangosteen extract and the fermented Mangosteen product, as it is or supported in maltodextrines, with preferably natural or synthetic cellulose derivatives in w/w percentage from 10% to 80%, or from 15% to 50%, wherein such cellulose derivatives are selected from: hydroxyl-propyl-methylcellulose, starch, methylcellulose, alginates, gelatine; or alternatively granulating Mangosteen extract and fermented Mangosteen product and coating the particles so obtained with natural or synthetic cellulose derivatives in percentage from 10% to 80%, or from 15% to 50%, wherein such cellulose derivatives are selected from: hydroxyl-propyl-methylcellulose, starch, methylcellulose, alginates, gelatine;
2) Coating of the granules obtained according to step 1) with gastroresistant agents preferably of natural origin, as vegetable waxes (carnauba wax, sandarac tree gum, arabic gum, shellac) or other natural layers like keratin, in a percentage w/w from 5% to 30%; or with synthetic coating agents, like cellulose acetophtalate, acrylic and meta-acrylic polymers, polyvinilacetate-phtalate;
3) Adding the composition with direct compression excipients like microcrystalline cellulose, magnesium stearate, talc, lactose, in percentage w/w between 3% and 20%, or around 15%, and mixing properly;
4) reducing the powder into tablets or capsule filling or sachet filling.

17. Process according to claim 14, comprising a step of direct compression of granules of step 1) or of the combination of steps 1) and 3), as described above, followed by the milling of the tablets obtained there from and then coating the dry particles according to step 2).

18. Process according to claim 11, wherein the said fermented Mangosteen product is obtained by a fermentation process that comprises fermenting whole fruits of Mangosteen, preferably homogenized in advance, with a starter, preferably Saccaromyces Boulardii, at a temperature of 35-55° C. for 2-4 months.

19. Process according to claim 11, wherein, instead of a fermented Mangosteen product, oligosaccharides in amounts ranging from 5 to 50 w/w % are used.

20. Pharmaceutical or veterinary formulation comprising the composition of claim 1.

21. Pharmaceutical or veterinary formulation according to claim 20 for use as antioxidant, antihistaminic, anti-inflammatory or anti-microbial agent.

22. Food or dietary supplement for humans or animals comprising the composition according to claim 1.

23. Cosmetic formulation comprising the composition of claim 1.
